(19) United States
(12) Patent Application Publication
(10) Pub. No.: US 2003/0040713 A1
(43) Pub. Date: Feb. 27, 2003

(54) Syringe device

(76) Inventors: Wen-Pin Wang, Taipei (TW); Te-Fa Lin, Taichung City (TW)

Correspondence Address: Hart, Baxley, Daniels & Holton, Fifth Floor, 59 John Street, New York, NY, 10038, US

(21) Appl. No.: 09/793,808

(22) Filed: Feb. 26, 2001

(51) Int. Cl. A61M 5/00

(52) U.S. Cl. 604/187; 604/192

(57) ABSTRACT

A syringe device includes a barrel in which a stopper connected to a plunger is movably received. An engaging member is connected to a first end of the barrel and has a protrusion extending therefrom so as to be connected to a needle cannula. A hole is defined in the engaging member and the stopper has an engaging part so as to be engaged with the hole to connect the engaging member and the stopper. The plunger has a thumb rest and a stop extends from the thumb rest. A notch is defined in the plunger and the stop is received in the notch so as to allow the plunger to be completely pushed in the barrel.

### FIELD OF THE INVENTION

The present invention relates to a syringe device wherein the plunger has a stop on the thumb rest and which will contact the finger flange of the barrel to prevent the plunger from completely being merged in the barrel.

### BACKGROUND OF THE INVENTION

FIG. 1 shows a conventional syringe device and includes a barrel 10 with an engaging tube 12 extending from an end of the barrel 10. A stopper 17 is movably received in the barrel 10 and connected to a plunger 15 by a connection member 16 which is securely inserted into the stopper 17. A locking tip 180 is mounted to the engaging tube 12 and a needle cannula 181 is connected to the locking tip 180. Medicine in the interior 11 of the barrel 10 can be injected out from the needle cannula 181 by pushing the plunger 15 toward the engaging tube 12. A cap can be mounted to the locking tip to enclose the needle cannula 181. After being used, the syringe device has to be discarded and the needle cannula 181 together with the locking tip 180 are removed from the engaging tube 12. However, it is dangerous that when mounting the cap to enclose the needle cannula 181, the user could be cut by the needle cannula 181 and is contaminated by the patient's blood on the needle cannula 181.

### SUMMARY OF THE INVENTION

In accordance with one aspect of the present invention, there is provided a syringe device and comprising a barrel having an engaging member connected to a first end of the barrel and a finger flange extending radially outward from a second end of the barrel. The engaging member has a protrusion extending therefrom and a passage defined through the protrusion. A hole defined in the engaging member and communicates with the passage. A first annular lip extends radially inward from an inner periphery of the hole. A stopper has a contacting flange and an engaging part extends from the contacting flange. A locking plate extends the engaging part and a groove is defined in a shank of the stopper. A biasing member is biased between the stopper and the engaging member.

A plunger has a recess defined in a first end thereof and a second annular lip extends from an inner periphery of the recess. The second annular lip is engaged with the groove to receive the shank of the stopper in the recess. A thumb rest extends radially outward from a second end of the plunger and a stop extends from the thumb rest and toward the first end of the plunger. A notch is defined in the plunger and located beside the stop.

The primary object of the present invention is to provide a syringe device wherein the thumb rest has a stop to prevent the plunger being connected to the needle engaging member before sucking medicine in the barrel.

These and further objects, features and advantages of the present invention will become more obvious from the following description when taken in connection with the accompanying drawings which show, for purposes of illustration only, several embodiments in accordance with the present invention.

### BRIEF DESCRIPTION OF THE DRAWINGS

FIG. 1 is a cross-sectional view to show a conventional syringe device;

FIG. 2 is an exploded view to show a syringe device of the present invention;

FIG. 3 is a cross-sectional view to show the plunger is stopped by the finger flange of the barrel of the syringe device of the present invention;

FIG. 4 is a cross-sectional view to show the plunger is pulled away the barrel of the syringe device of the present invention;

FIG. 5 shows that medicine is sucked in the barrel by pulling the plunger of the syringe device of the present invention;

FIG. 6 shows that air in the barrel is pushed through the needle cannula by slightly pushing the plunger of the syringe device of the present invention;

FIG. 7 shows that the air in the barrel is completely expelled out from the barrel of the syringe device of the present invention;

FIG. 8 shows that the plunger is pushed into the barrel and connected to the engaging member of the syringe device of the present invention;

FIG. 9 shows that the plunger is pulled after the syringe is finished and the shank of the plunger is broken;

FIG. 10 shows that the needle cannula is pulled in the barrel and bent;

FIG. 11 is an exploded view to show another embodiment of the syringe device of the present invention;

FIG. 12 is an exploded view to show yet another embodiment of the syringe device of the present invention;

FIG. 13 is an exploded view to show an embodiment of the plunger of the syringe device of the present invention;

FIG. 14 is an exploded view to show another embodiment of the stopper on the plunger of the syringe device of the present invention, and

FIG. 15 is a cross sectional view to show the stopper as shown in FIG. 14 is stopped by the finger flange of the barrel of the syringe device of the present invention.

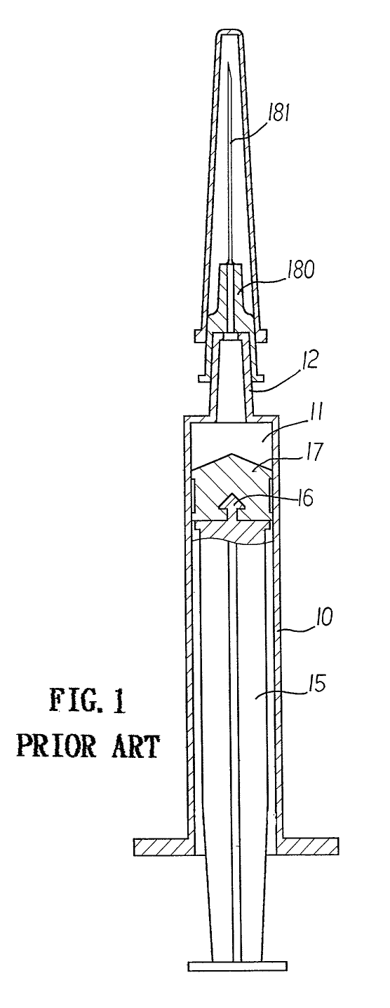

### DETAILED DESCRIPTION OF THE PREFERRED EMBODIMENTS

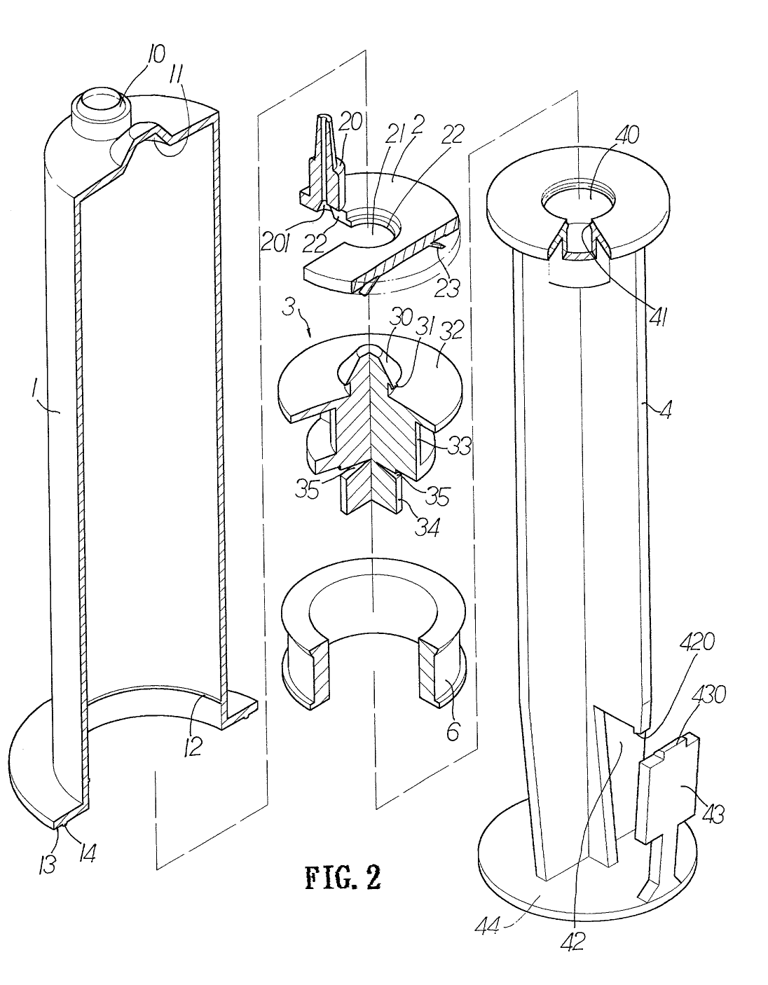

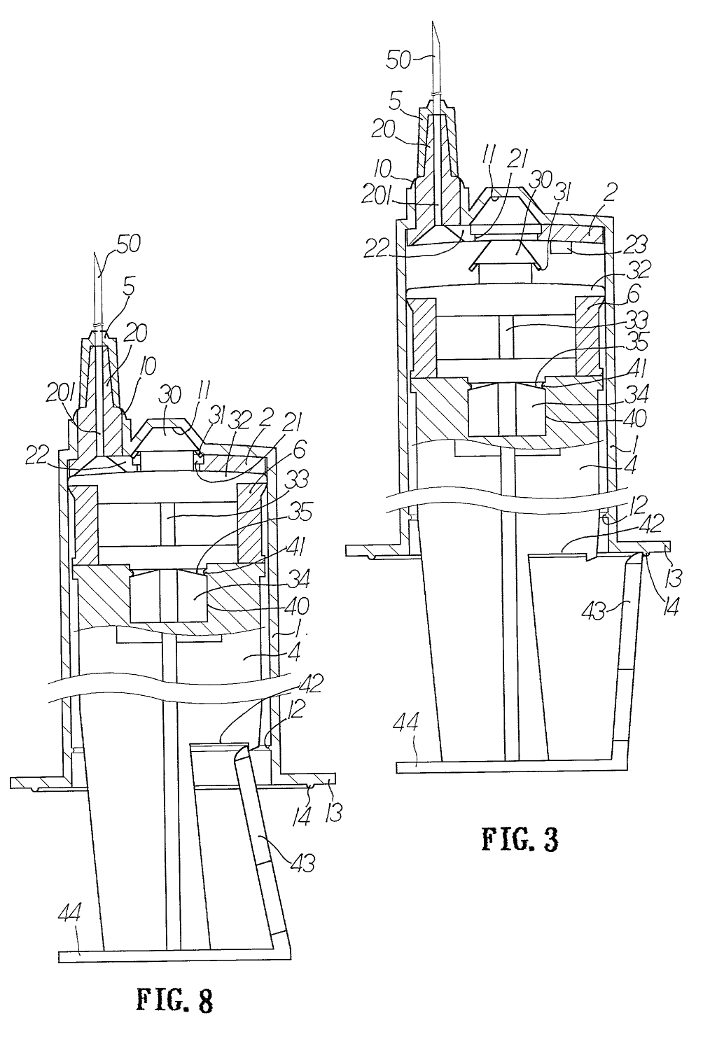

Referring to FIGS. 2 and 3, the syringe device of the present invention comprises a barrel 1 having a first end which is a closed end and a recess 11 is defined in inside of the closed end. A tube 10 extends from the closed end and communicates with an interior of the barrel 1. An engaging member 2 has a hole 21 defined therethrough and a protrusion 20 extends from the engaging member 2. A passage 201 is defined through the protrusion 20 and the protrusion 20 extends through the tube 10 so that a needle unit 5 with a needle cannula 51 are mounted to the the protrusion 20. A first annular lip 22 extends radially inward from an inner periphery of the hole 21. Two biasing members 23 respectively extend from the engaging member 2. A finger flange 13 extends radially outward from a second end of the barrel 1 and a ridge 14 extends from the finger flange 13 of the barrel 1.

A stopper 3 has a contacting flange 32 and a cone-shaped engaging part 30 extends from the contacting flange 32. A locking plate 31 extends from the engaging part 30 and a groove 35 is defined in a shank of the stopper 3 to form a first section 33 and a second section 34. A seal 6 is mounted to the first section 33.

A plunger 4 has a recess 40 defined in a first end thereof and a second annular lip 41 extends from an inner periphery of the recess 40. The second section 34 of the stopper 3 is engaged with the recess 40 of the plunger 4 and the second annular lip 41 is engaged with the groove 35. A thumb rest 44 extends radially outward from a second end of the plunger 4 and a stop 43 extends from the thumb rest 44 and toward the first end of the plunger 4. A notch 42 is defined in the plunger 4 and located beside the stop 43. A hook 420 extends from an inner periphery of the notch 32 and a tongue 430 extends from the stop 43.

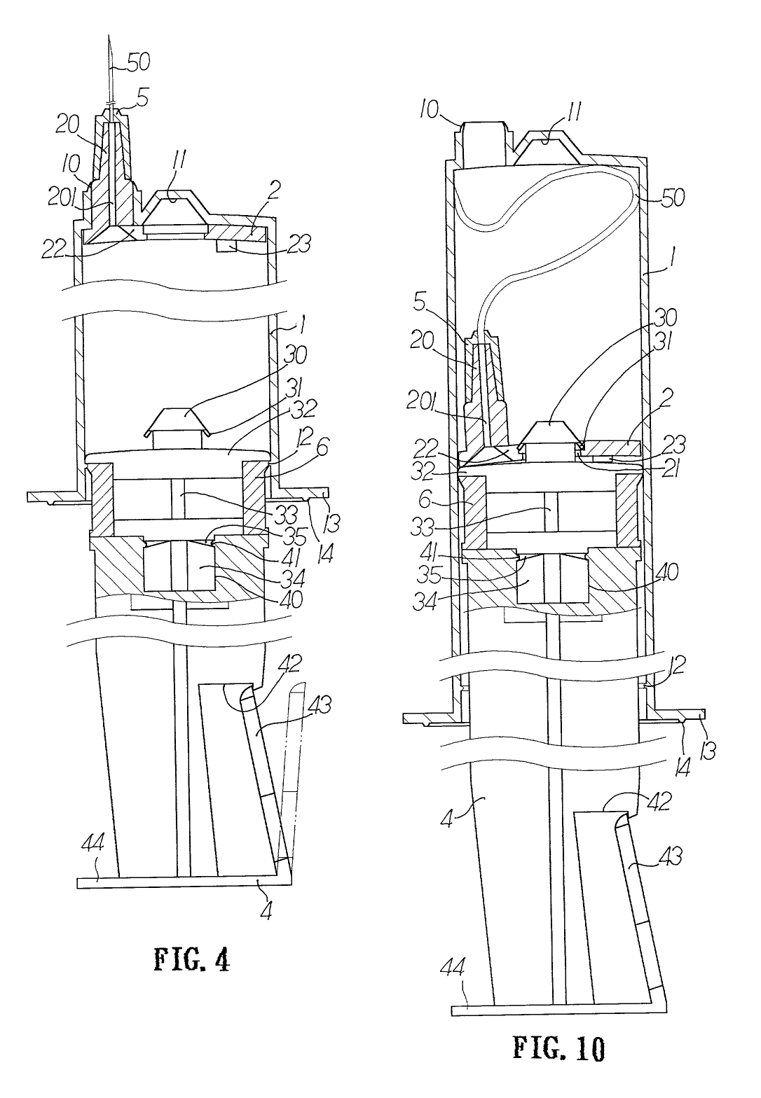

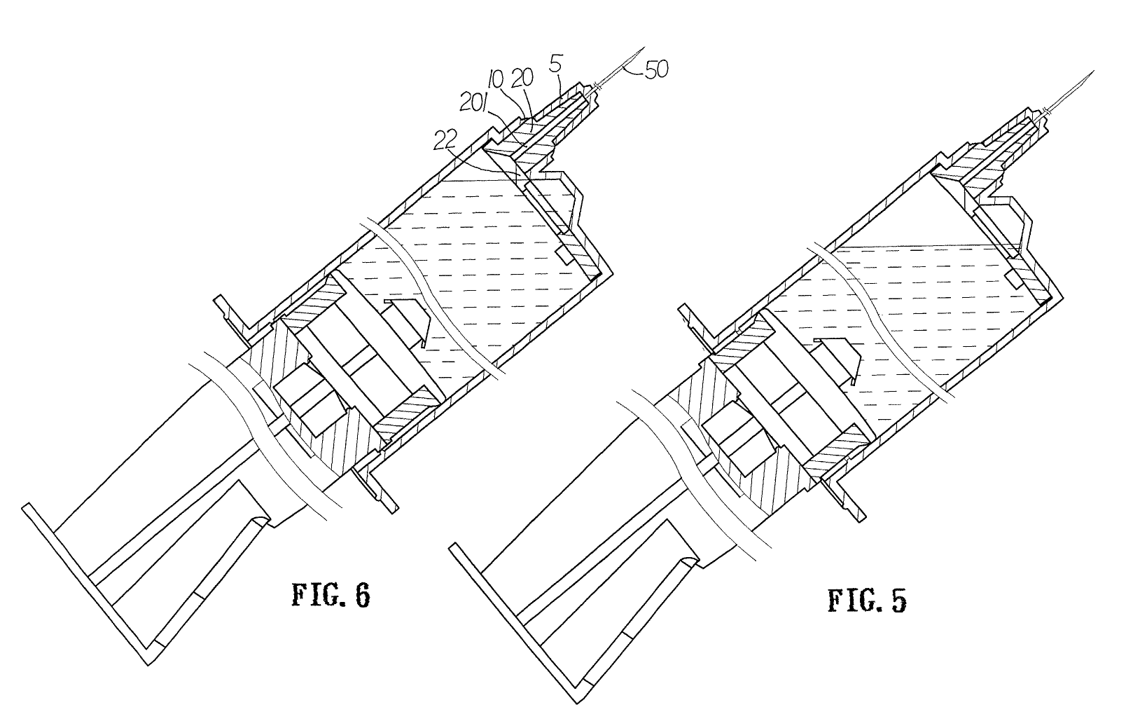

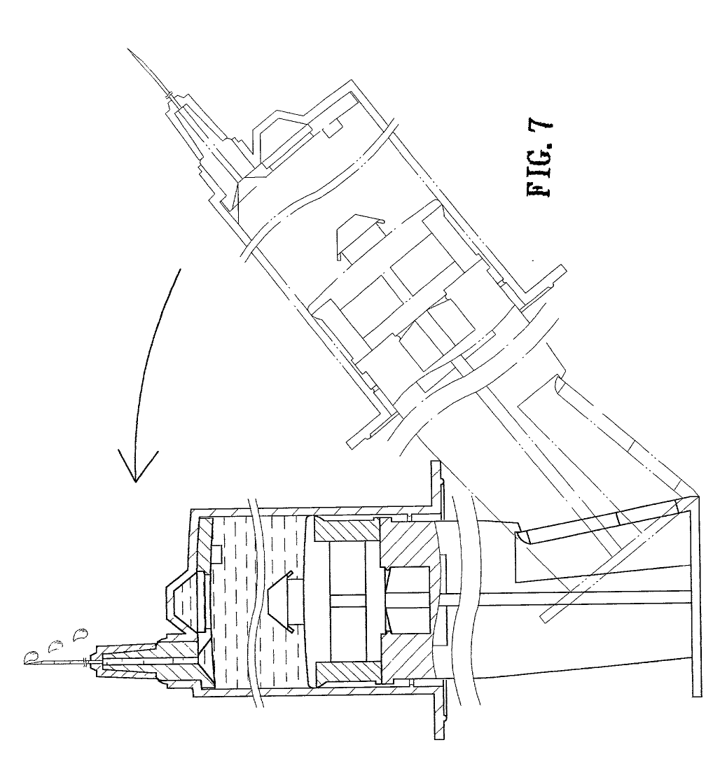

As shown in FIGS. 4 to 6, when sucking medicine in the barrel 1, the plunger 4 is pulled and medicine is sucked in the barrel 1 via the needle cannula 50. Before injecting the medicine, the air in the barrel 1 is pushed out from the needle cannula 50 by pushing the plunger 4 into the barrel 1. The barrel 1 is tilted to let the air be expelled from the needle cannula 50 by pushing the plunger 4 till the barrel is oriented upstraight as shown in FIG. 7 to completely expel the air from the barrel 1.

As shown in FIG. 8, the medicine is pushed from the needle cannula 50 by pushing the plunger 4 and the stop 43 has to be pushed to let the tongue 430 be engaged with the hook 420. The inward pushed stopper 43 allows the plunger 4 to be completely inserted into the barrel 1 and the cone-shaped engaging part 30 is then engaged with the hole 21, and the locking plate 31 is engaged with the first annular lip 22 of the hole 21 to connect the engaging member 2 and the stopper 3. At this position, all the medicine is pushed out from the needle cannula 50. It is to be noted that, as shown in FIG. 3, if the stop 43 is not engaged with the notch 42, the plunger 4 cannot be completely inserted into the barrel 1 and the tongue 430 is stopped by the ridge 14. Therefore, the stopper 3 will not engaged with the engaging member 2.

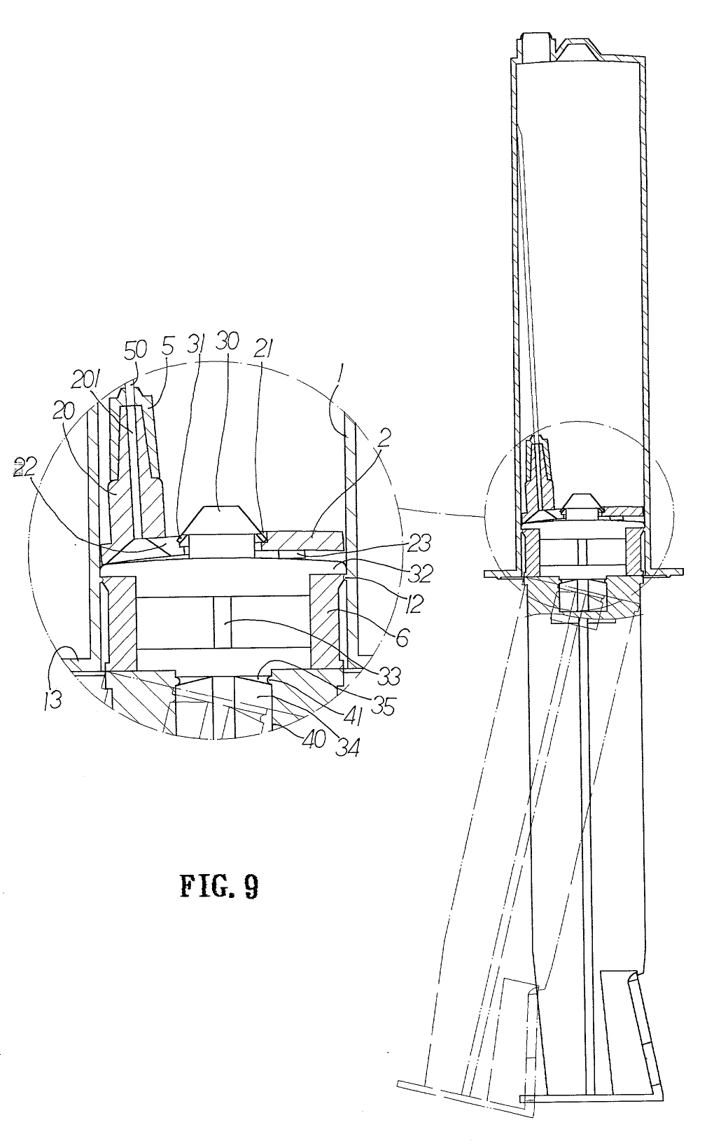

After the medicine runs out from the barrel 1, the plunger 4 is pulled back as shown in FIG. 9 and the engaging member 2 together with the needle unit 5 is pulled into the barrel 1. An indexing flange 12 extends radially inward from an inner periphery of the second end of the barrel 1 and the stopper 3 is stopped by the indexing flange 12. At this position, the plunger 4 can be easily broken at the position of the groove 35. As shown in FIG. 10, when the engaging member 2 is removed from the closed end of the barrel 1, the biasing plates 23 kick the stopper 3 and let the needle cannula 50 be inclined in the barrel 1. The needle cannula 50 is then easily bent by pushing the plunger 4 slightly to let the needle cannula 50 be clamped and bulked between the closed end of the barrel 1 and the engaging member 2.

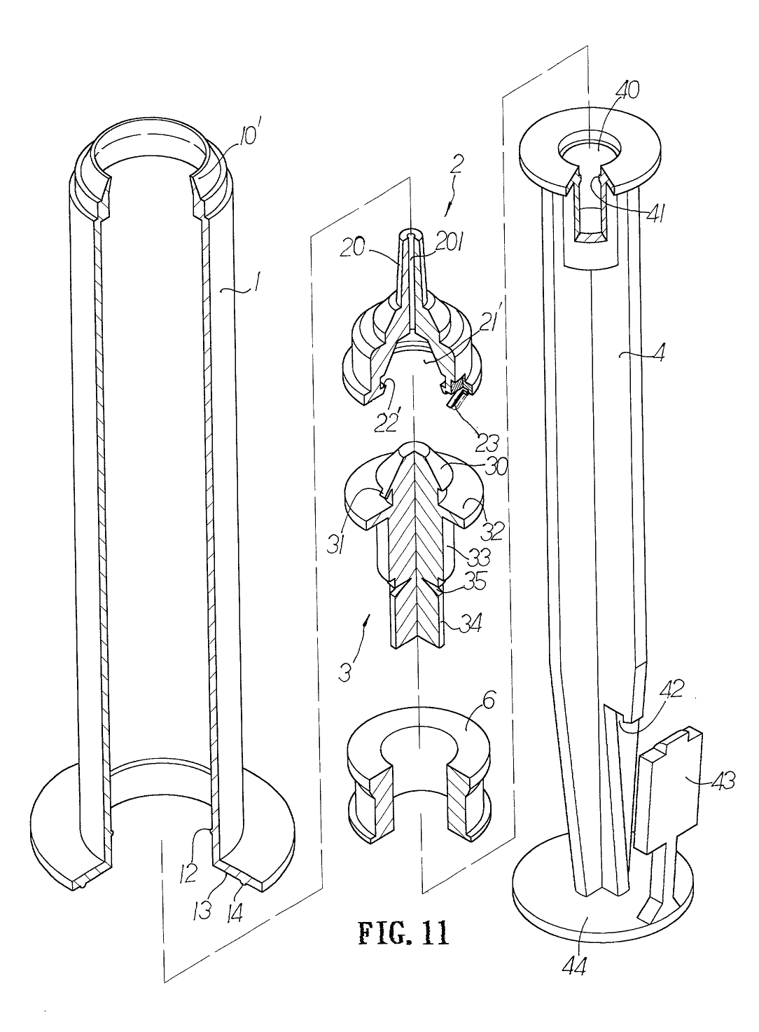

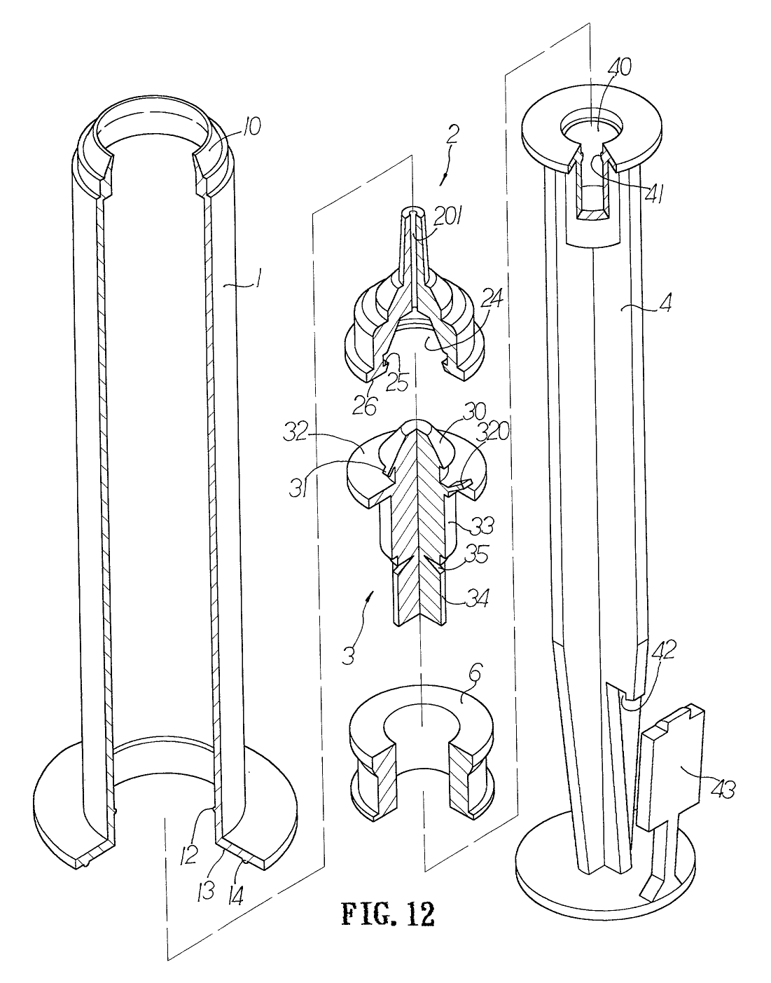

FIG. 11 shows that the first end of the barrel 1 has a central tube 10′ and the engaging member 2 is directly engaged with the central tube 10′. The hole 21′ is defined in the engaging member 2 and has the first annular lip 22 extending radially inward from an inner periphery of the hole 21′ so as to be engaged with the cone-shaped engaging part 30 as described hereinabove. FIG. 12 shows that the biasing member 30 extend from the contacting flange 32 of the stopper 3 and has the same function of the biasing member 23 extending from the engaging member 2 as shown in FIG. 11.

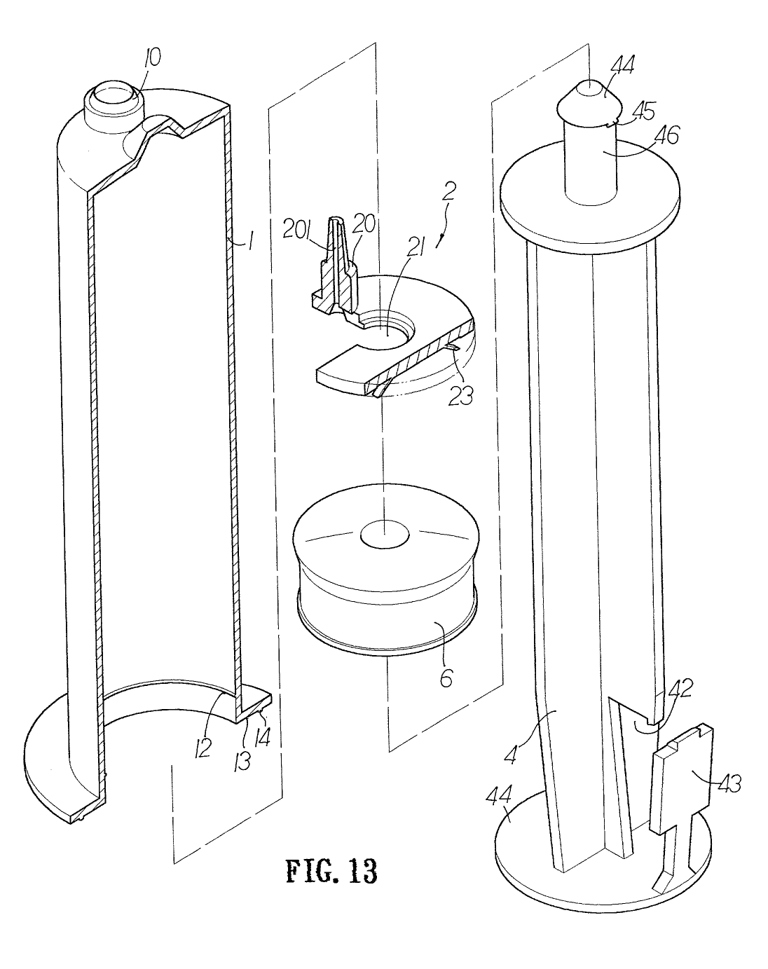

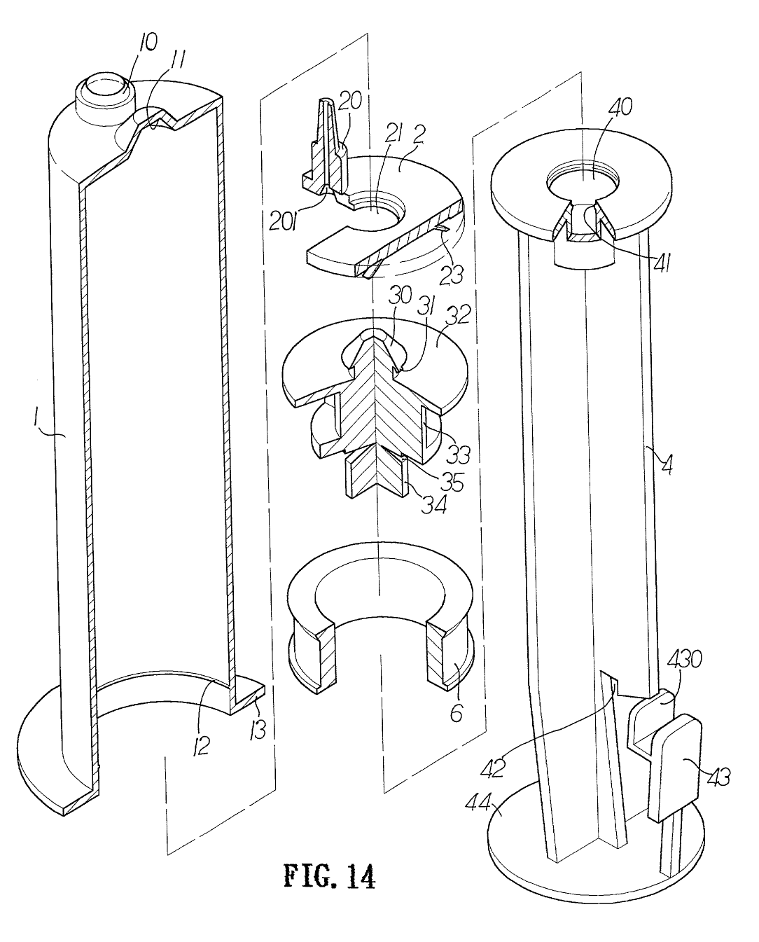

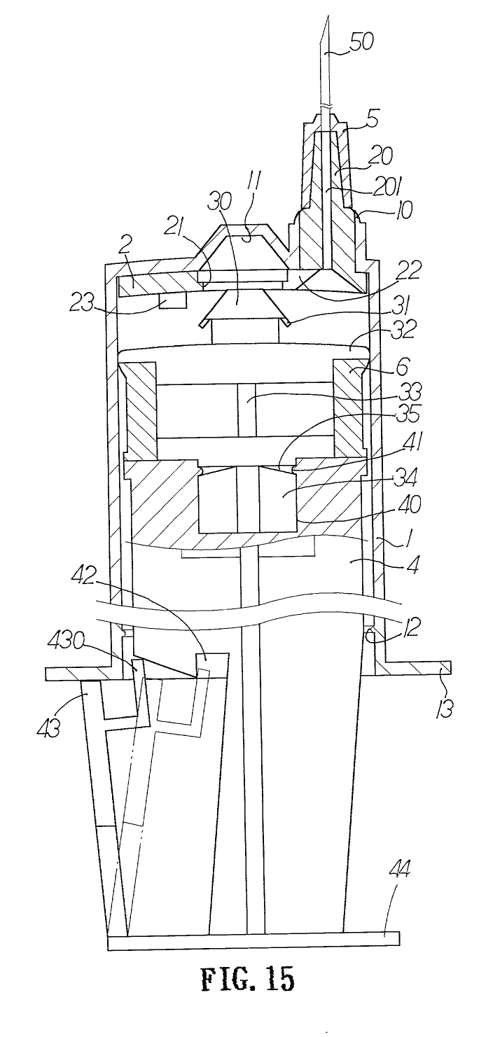

FIG. 13 shows that the stopper can be made integrally with the plunger 4. The stopper includes a shank 46 and a cone-shaped head 44 on the shank 46. A locking plate 45 extends from the lower edge of the cone-shaped head 44. FIGS. 14 and 15 show that the stop 43 may have an L-shaped extension 430 extending from a side of the stop 43 and the L-shaped extension 430 is to be engaged with the notch 42.

While we have shown and described various embodiments in accordance with the present invention, it should be clear to those skilled in the art that further embodiments may be made without departing from the scope and spirit of the present invention.

What is claimed is:

1. A syringe device comprising:
a barrel having an engaging member connected to a first end of said barrel and a finger flange extending radially outward from a second end of said barrel, said engaging member having a protrusion extending therefrom and a passage defined through said protrusion, a hole defined in said engaging member and communicating with said passage, a first annular lip extending radially inward from an inner periphery of said hole;
a stopper having a contacting flange and an engaging part extending from said contacting flange, a locking plate extending from said engaging part and a groove defined in a shank of said stopper, a biasing member biased between said stopper and said engaging member, and
a plunger having a recess defined in a first end thereof and a second annular lip extending from an inner periphery of said recess, said second annular lip engaged with said groove to receive said shank of said stopper in said recess, a thumb rest extending radially outward from a second end of said plunger and a stop extending from said thumb rest and toward said first end of said plunger, a notch defined in said plunger and located beside said stop.

2. The device as claimed in claim 1 further comprising a hook extending from an inner periphery of said notch so as to engage with said stop.

3. The device as claimed in claim 2 further comprising a tongue extending from said stop which is engaged with said hook of said notch.

4. The device as claimed in claim 1 further comprising a ridge extending from said finger flange of said barrel.

5. The device as claimed in claim 1 further comprising an indexing flange extending radially inward from an inner periphery of said second end of said barrel.

6. The device as claimed in claim 1 wherein said first end of said barrel is a closed end and a recess is defined in inside of said closed end, a tube extending from said closed end and communicating with an interior of said barrel, said protrusion extending through said tube.
